(19) United States
(12) Patent Application Publication
(10) Pub. No.: US 2023/0072174 A1
(43) Pub. Date: Mar. 9, 2023

(54) DEVICES, SYSTEMS, AND METHODS FOR VISUALIZING, ACCESSING AND PERFORMING INTERVENTIONS INVOLVING THE THORACIC DUCT

(71) Applicant: Daniel A. Crawford, St. Louis, MO (US)

(72) Inventor: Daniel A. Crawford, St. Louis, MO (US)

(21) Appl. No.: 17/939,787

(22) Filed: Sep. 7, 2022

Related U.S. Application Data
(60) Provisional application No. 63/241,396, filed on Sep. 7, 2021.

(51) Int. Cl. A61B 8/08; A61B 8/00

(52) U.S. Cl. CPC A61B 8/0891; A61B 8/445; A61M 25/0084

(57) ABSTRACT

The present disclosure relates generally to devices, systems, and methods for minimally invasive medical treatment, methods of diagnosing and treating diseases, and to visualizing, accessing, and perform interventions involving the thoracic duct.

### CROSS REFERENCE TO RELATED APPLICATIONS

The present invention claims priority to and the benefit of U.S. Provisional Patent App. No. 63/241,396 filed Sep. 7, 2021, entitled “METHODS OF DIAGNOSING, ACCESSING, AND PERFORMING INTERVENTIONS INVOLVING THE THORACIC DUCT,” the entire disclosure of which is hereby incorporated herein by reference in its entirety and made a part hereof. Any and all priority claims identified in the Application Data Sheet, or any corrections thereto, are hereby incorporated by reference under 37 CFR 1.57.

### BACKGROUND

Field: This technology as disclosed herein relates generally to devices, systems, and methods for visualizing, accessing and performing interventions involving the thoracic duct, including diagnosing and treating diseases.

Background: The thoracic duct is the largest lymphatic structure in the human body, most commonly arising from the cisterna chyli along the lower thoracic spine/upper lumbar spine and running cranially along the spine until the superior chest where it courses toward the left to enter the venous circulation near the venous angle (the junction between the internal jugular and subclavian veins). Variability in thoracic duct anatomy is common, including branching into multiple ducts with multiple anastomoses near the venous angle and absence of the cisterna chyli. The thoracic duct serves as a conduit for most of the lymph produced throughout the body.

Present efforts of accessing the thoracic duct are limited in that they focus on access at the distal end of the thoracic duct near the natural opening at the junction with the left internal jugular vein and the left subclavian vein. Related art describes accessing the thoracic duct at its entrance into the internal jugular vein/subclavian vein, from the internal jugular vein or subclavian vein directly into the thoracic duct, from the inferior vena cava directly into the cisterna chyli, percutaneously (through the skin) into the cervical thoracic duct, and percutaneously through the abdomen into the cisterna chyli.

As such, current methods to access the thoracic duct are technically challenging, operator dependent, time consuming, and frequently unsuccessful. In addition, reliable imaging of the thoracic duct is expensive, time consuming, and not widely available. Unfortunately, there was previously no satisfactory device, method, or system to image, achieve access to, or perform interventions involving the thoracic duct.

It is with these observations in mind, among others, that various aspects of the present disclosure were conceived and developed.

### SUMMARY

The devices, systems, and methods of the present disclosure have several features and implementations, no single one of which is solely responsible for its desirable attributes. Without limiting the scope of this invention as expressed by the claims which follow, its more prominent features will now be discussed briefly. After considering this discussion, and particularly after reading the section entitled “Detailed Description,” one will understand how the features of this disclosure provide several advantages over other devices, systems, and methods.

Implementations and embodiments of the present invention relate generally to minimally invasive medical treatment methods of diagnosing and treating diseases, specifically to visualizing, accessing, and performing interventions involving the thoracic duct.

Certain implementations of the technology as disclosed herein include a method of diagnosing the thoracic duct and/or thoracic duct pathology by inserting an intravascular imaging device in a vessel adjacent to the thoracic duct. Certain other implementations of the technology as disclosed herein include a method of accessing the thoracic duct by inserting a catheter/device in a vessel adjacent to the thoracic duct and accessing the thoracic duct using this catheter/device. Certain additional implementations of the technology as disclosed herein include a method of performing an intervention involving the thoracic duct by utilizing the catheter/device in the vessel adjacent to the thoracic duct.

Accordingly, several advantages of one or more aspects of the disclosed systems, devices, and methods to visualize and intervene on the thoracic duct include: simpler and easier to learn given a single access site, more repeatable given the proximity of the vein and thoracic duct, and faster procedures without having to wait for thoracic duct opacification prior to visualization or access.

The features, functions, and advantages that have been discussed can be achieved independently in various implementations or can be combined in yet other implementations further details of which can be seen with reference to the following description and drawings.

These and other advantageous features of the present technology as disclosed will be in part apparent and in part pointed out herein below.

### BRIEF DESCRIPTION OF THE DRAWINGS

For a better understanding of the present technology as disclosed, reference may be made to the accompanying drawings in which:

FIG. 1 depicts a flow diagram illustrating a method of performing a procedure to image the thoracic duct according to one implementation.

FIG. 2 depicts a flow diagram illustrating a method of accessing the thoracic duct according to one implementation.

FIG. 3 depicts a flow diagram illustrating a method of performing an intervention involving the thoracic duct using thoracic duct access according to one implementation.

FIG. 4. depicts a flow diagram illustrating a method of performing an intervention on the thoracic duct according to one implementation.

FIG. 5 depicts an anatomical diagram depicting the relevant anatomy for an implementation of the invention.

FIGS. 6A to 6C depict various aspects according to an implementation of visualizing, accessing and treating the thoracic duct via an adjacent vein.

FIGS. 7A and 7B depicts certain implementations of devices, systems, and methods for accessing and treating the thoracic duct.

FIGS. 8A and 8B depicts certain implementations of devices, systems, and methods for accessing and treating the thoracic duct.

FIGS. 9A and 9B depict certain implementations of devices, systems, and methods for accessing and treating the thoracic duct that allows for a separate needle guide device that can fit onto an imaging catheter.

While the technology as disclosed is susceptible to various modifications and alternative forms, specific implementations thereof are shown by way of example in the drawings and will herein be described in detail. It should be understood, however, that the drawings and detailed description presented herein are not intended to limit the disclosure to the particular implementations as disclosed, but on the contrary, the intention is to cover all modifications, equivalents, and alternatives falling within the scope of the present technology as disclosed and as defined by the appended claims.

### DETAILED DESCRIPTION

Implementation(s) of the invention are discussed below with reference to the Figures. However, those skilled in the art will readily appreciate that the detailed description given herein with respect to these figures is for explanatory purposes as the invention extends beyond these limited implementations. For example, it will be appreciated that those skilled in the art will, in light of the teachings of the present invention, recognize a multiplicity of alternate and suitable approaches, depending upon the needs of the particular application, to implement the functionality of any given detail described herein, beyond the particular implementation choices described and shown. That is, there are modifications and variations of the invention that are too numerous to be listed but that all fit within the scope of the invention.

Singular words should be read as plural and vice versa and masculine as feminine and vice versa, where appropriate, and alternative implementations do not necessarily imply that the two are mutually exclusive. It is to be further understood that the present invention is not limited to the particular methodology, material, use, or application described herein, as these can vary. It is also to be understood that the terminology used herein is used for the purpose of describing particular implementations and embodiments only and is not intended to limit the scope of the present invention.

It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and can include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that can be construed to express approximation should be so understood unless the context clearly dictates otherwise.

It will be further understood that use of the word “can” and/or “may” will be understood to refer to the active, and enabling, dictionary meanings of “is able,” “be able”, “to know,” “be able to through acquired knowledge or skill,” “to know how to do something,” and/or “to have the ability to do something”; and not understood to intend a sense of “maybe” or permissiveness.

Reference in the specification to “one embodiment” or “an embodiment”; “one implementation” or “an implementation” means that a particular feature, structure, or characteristic described in connection with the embodiment or implementation is included in at least one embodiment or implementation of the invention. The appearances of the phrase “in one embodiment,” or “in an embodiment,” or “in one implementation,” or “in an implementation” in various places in the specification are not necessarily all referring to the same embodiment or the same implementation, nor are separate or alternative embodiments or implementations mutually exclusive of other embodiments or implementations.

[RESERVED]

[RESERVED]

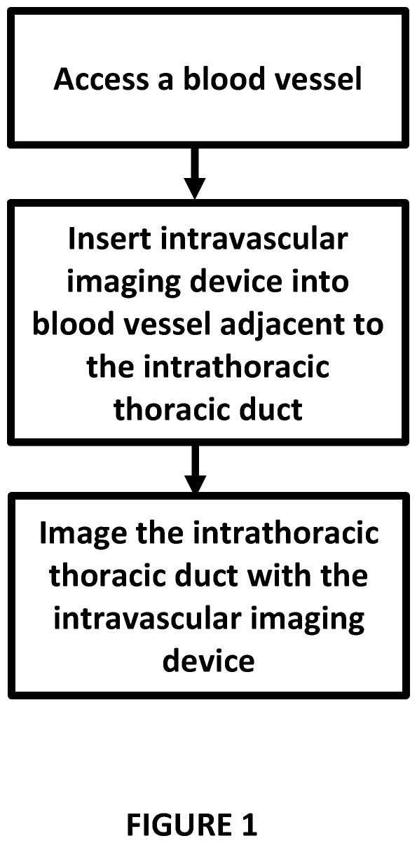

According to the implementation(s) of the present technology as disclosed, various views are illustrated in FIGS. 1-9 and like reference numerals are being used consistently throughout to refer to like and corresponding parts of the technology for all of the various views and figures of the drawing. Also, please note that the first digit(s) of the reference number for a given item or part of the technology should correspond to the FIG. number in which the item or part is first identified.

Described herein are novel devices, systems, and methods which are not described in the current literature. These novel devices, systems, and methods in certain embodiments comprise inserting a device or devices into an azygos vein, a hemiazygos vein, and/or or one of the tributaries of either or both.

The uniqueness of the presently disclosed devices, systems, and methods pertains to imaging, diagnosing, accessing, and/or performing interventions on the thoracic duct or cisterna chyli from the azygos vein, hemiazygos vein, or one of their tributaries. These devices, systems, and methods have not been described in other patents or the literature.

Any number of imaging and treatments on the thoracic duct can be performed using embodiments of the present invention. These include but are not limited to: diagnostic imaging of the thoracic duct (intravascular ultrasound, optical coherence tomography); removing lymphatic fluid; instilling fluid into thoracic duct (including contrast for diagnostic purposes); embolizing the thoracic duct; creating a shunt between the thoracic duct and a vein; and many others.

[RESERVED]

Reference may be made to the following, each hereby incorporated by reference herein as if set forth in its entirety:
“Atlas of Anatomy”, Thieme; 2nd edition (Apr. 9, 2012) ISBN-10: 1604067454; pages 78-81 “Veins of the Thoracic Cavity,” “Lymphatics of the Thoracic Cavity”
“Netter Atlas of Human Anatomy: Classic Regional Anatomy Approach”, 4, 227-298.e12; Plate 211 Anterior Thoracic Wall: Internal View; Plate 213 Veins of Thoracic Wall; Plate 215 Diaphragm: Thoracic Surface; Plate 220 Great Vessels of Mediastinum; Plate 237 Mediastinum: Cross Section; Plate 265 Cross Section of Thorax at T4/T5 Disc Level
https://academic.oup.com/ejcts/article/53/5/1093/4728648
https://www.sciencedirect.com/science/article/abs/pii/S0899707116302078
https://radiopaedia.org/articles/thoracic-duct?lang=us
https://www.ncbi.nlm.nih.gov/pmc/articles/PMC3140251/#!po=1.08696
https://www.kenhub.com/en/library/anatomy/azygos-vein
“Lymphatic Intervention for Various Types of Lymphorrhea: Access and Treatment”, Masanori Inoue, Seishi Nakatsuka, Hideki Yashiro, Masashi Tamura, Yohsuke Suyama, Jitsuro Tsukada, Nobutake Ito, Sota Oguro, and Masahiro Jinzaki, RadioGraphics 2016 36:7, 2199-2211
Itkin, M., Nadolski, G. J. “Modern Techniques of Lymphangiography and Interventions: Current Status and Future Development.” Cardiovasc Intervent Radiol 41, 366-376 (2018). https://doi.org/10.1007/s00270-017-1863-2
Kariya, S., Nakatani, M., Ueno, Y. et al. “Transvenous Retrograde Thoracic Ductography: Initial Experience with 13 Consecutive Cases.” Cardiovasc Intervent Radiol 41, 406-414 (2018). https://doi.org/10.1007/s00270-017-1814-y
Novelli P M, Chan E G, Frazier A A, Villa Sanchez M. “Interventional Therapies for Thoracic Duct Injury and Intractable Chylothorax.” J Thorac Imaging. 2019 July;34(4):258-265. doi: 10.1097/RTI.0000000000000422. PMID: 31206455.
Sato, Y., Tanaka, Y., Imai, T. et al. “Chylothorax after esophagectomy treated with inguinal intranodal lymphangiography and transvenous retrograde thoracic duct embolization.” Clin J Gastroenterol 14, 969-974 (2021). https://doi.org/10.1007/s12328
“Lymphatic Interventional Treatment for Chyluria via Retrograde Thoracic Duct Access,” Hur, Saebeom et al. Journal of Vascular and Interventional Radiology, Volume 32, Issue 6, 896-900.
Jun, H., Hur, S., Jeong, Y. S. et al. “Thoracic duct embolization in treating postoperative chylothorax: does bail-out retrograde access improve outcomes?.” Eur Radiol 32, 377-383 (2022). https://doi.org/10.1007/s00330-021-08145-9.

[RESERVED]

Imaging of the thoracic duct is difficult and evaluation for thoracic duct leaks is challenging. Currently, dynamic contrast enhanced magnetic resonance lymphangiography can be used to image the thoracic duct and evaluate for leaks. However, this involves instilling gadolinium contrast into groin lymph nodes and obtaining multiple magnetic resonance images over time to image the thoracic duct. This method is time consuming, expensive, and few medical centers can perform this imaging modality.

Since at least the 1960s, surgical interventions performed on the thoracic duct include thoracic duct to venous anastomoses for palliation of cirrhosis, lymphatic drainage for heart failure, lymph drainage for lymphocyte depletion and immunosuppression, and thoracic duct ligation for chyle leaks and chylous effusions. While many of these surgeries demonstrated beneficial results, most have not been widely disseminated due to the difficulty and invasiveness of the operations.

In the 1990s a new minimally invasive technique was developed to access the thoracic duct, which involves instilling contrast into lower extremity lymphatics/groin lymph nodes, allowing this contrast to reach the cisterna chyli, and accessing the cisterna chyli with a small gauge needle under fluoroscopic guidance with an anterior transabdominal approach. Upon entry of the needle into the cisterna chyli, a guidewire is passed through the needle into the thoracic duct and interventions can be performed by those skilled in the art. For example, embolization of the thoracic duct for treatment of a chyle leak. However, this procedure has multiple drawbacks. First, it is technically challenging, with success rates reported in the literature ranging less than 75%. Many interventionalists are unable to perform this procedure due to its technical difficulty. Second, it is time consuming. Contrast instilled into the lower extremity lymphatics/groin lymph nodes can take up to half an hour or longer to reach the cisterna chyli and sometimes the cisterna chyli is never visualized or congenitally absent. If the cisterna chyli is visualized, there is a steep learning curve and significant operator variability in accessing the cisterna chyli from an anterior abdominal approach due to the posterior location and small caliber of the cisterna chyli. Procedures using this method can last longer than 3-4 hours just to obtain access to the thoracic duct. Oftentimes access to the thoracic duct can never be achieved.

Several additional minimally invasive techniques have been described to access the thoracic duct. However, these techniques have multiple drawbacks and do not allow consistent access to the thoracic duct. The cervical portion of the thoracic duct at the base of the neck can sometimes be accessed by inserting a small gauge needle into the thoracic duct under ultrasound guidance using an ultrasound transducer external to the patient. A guidewire is then passed through the needle into the thoracic duct. However, this approach is technically difficult with a steep learning curve and success rates of less than 50% reported in the literature. There is variability in the termination of the thoracic duct, oftentimes with multiple small caliber ducts that connect to the venous structures in the neck, and these small caliber ducts cannot be successfully cannulated due to their small size. A third technique for accessing the thoracic duct is via a transvenous approach, where a guidewire is introduced into the venous vasculature and attempts are made to pass the guidewire through the orifice of the thoracic duct near its anastomosis around the venous angle with fluoroscopic guidance. Small studies have shown limited success with this technique. As mentioned above, oftentimes there are multiple small caliber ducts that connect to the venous system that cannot be accessed using this technique. Additionally, there is typically a valve at the junction between the thoracic duct and vein that restricts retrograde flow and makes passage of a guidewire difficult.

[RESERVED]

Reference may be made to the following reference numerals as used herein:
510 -Right subclavian vein
520 -Right internal jugular vein
530 -Azygos vein orifice
540 -Azygos vein
550 -Superior vena cava
560 -Intrathoracic thoracic duct
570 -Left internal jugular vein
580 -Terminal thoracic duct at venous angle
590 -Left subclavian vein
610 -Intravascular imaging device
620 -Guidewire in the thoracic duct
630 -Catheter/device travelling from azygos vein into thoracic duct
640 -Azygos vein
650 -Intrathoracic thoracic duct

[RESERVED]

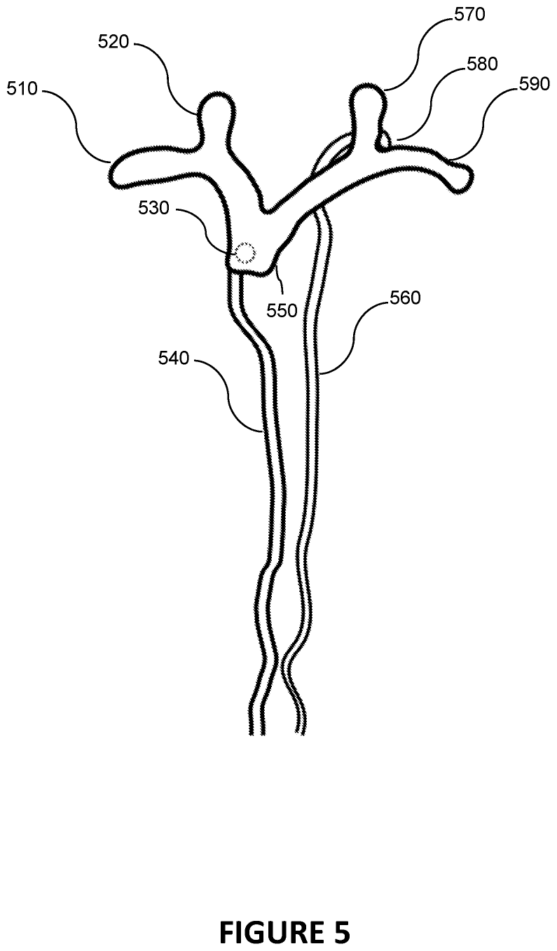

Relevant anatomy involving an implementation of the present invention is seen in FIG. 5. The right subclavian vein 510 joins the right internal jugular vein 520 and the left subclavian vein 590 joins the left internal jugular vein 570. These vessels join to form the superior vena cava 550. The azygos vein 540 originates off the superior vena cava at the azygos vein orifice 530. The intrathoracic thoracic duct 560 travels superiorly and the terminal thoracic duct 580 joins the venous system near the venous angle.

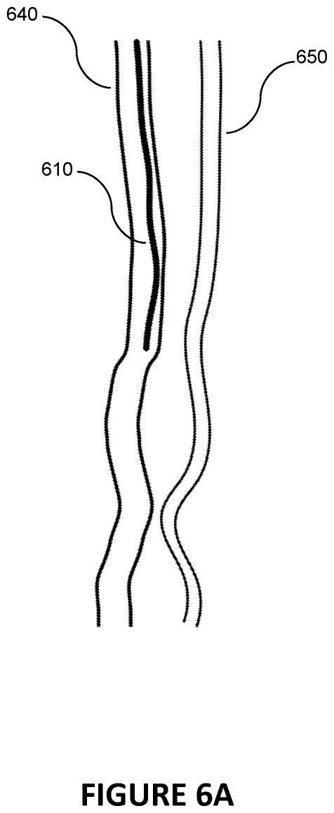

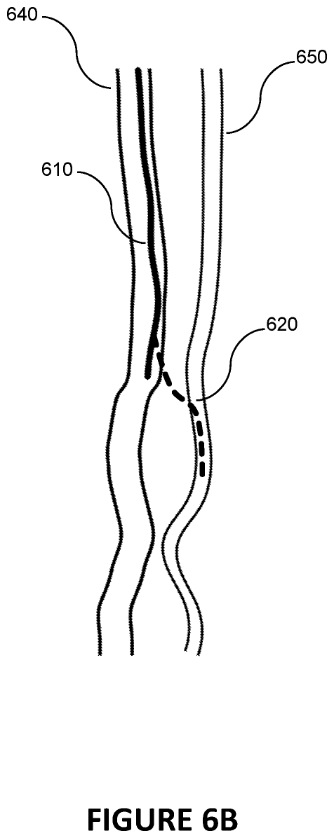

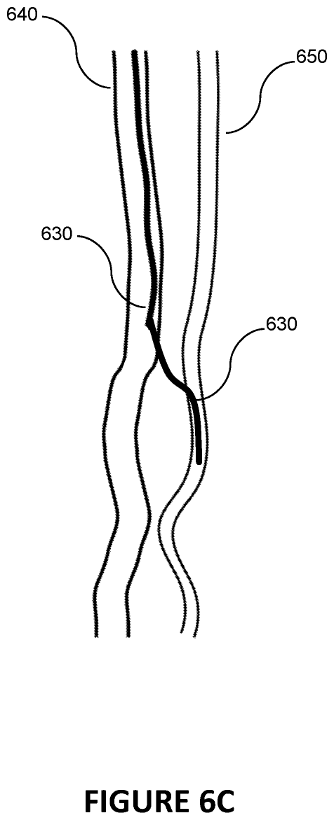

One implementation of imaging the thoracic duct is illustrated in FIG. 1 and FIG. 6A. After accessing a blood vessel, for example though not limited to limited to percutaneously into the right internal jugular vein, an intravascular imaging device is advanced into a vein adjacent to the thoracic duct. For example, an imaging catheter 610 can be advanced over a guidewire that was placed into the azygos vein 640 adjacent to the thoracic duct 650. After imaging catheter placement, imaging of the thoracic duct can be performed using an imaging device at the distal end of the imaging catheter. Imaging modalities include but are not limited to ultrasound, optical coherence tomography, or near-infrared spectroscopy.

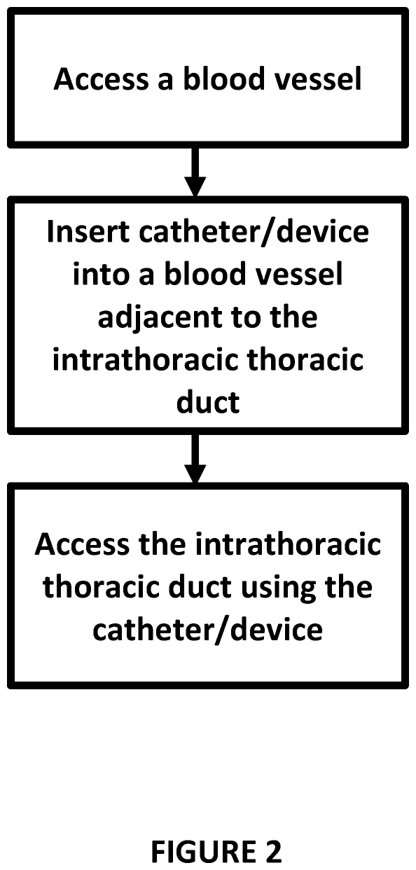

One implementation of accessing the thoracic duct is illustrated in FIG. 2 and FIG. 6B. After accessing a blood vessel, for example though not limited to percutaneously into the right internal jugular vein, a catheter/device is advanced into a vein adjacent to the thoracic duct. For example, a catheter/device 610 can be advanced over a guidewire that was placed into the azygos vein 640 adjacent to the thoracic duct 650. After catheter/device placement, a thoracic duct access needle can be advanced from the catheter/device and into the thoracic duct. This access can be achieved using the imaging catheter with an imaging device at the distal end of the catheter that allows for visualization of the thoracic duct and at least a portion of the access needle. After access needle placement, a guidewire 620 can be advanced into the thoracic duct 650. In another implementation, the access needle can curve such that it enters the thoracic duct pointed superiorly, allowing for easier guidewire placement that travels superiorly in the thoracic duct.

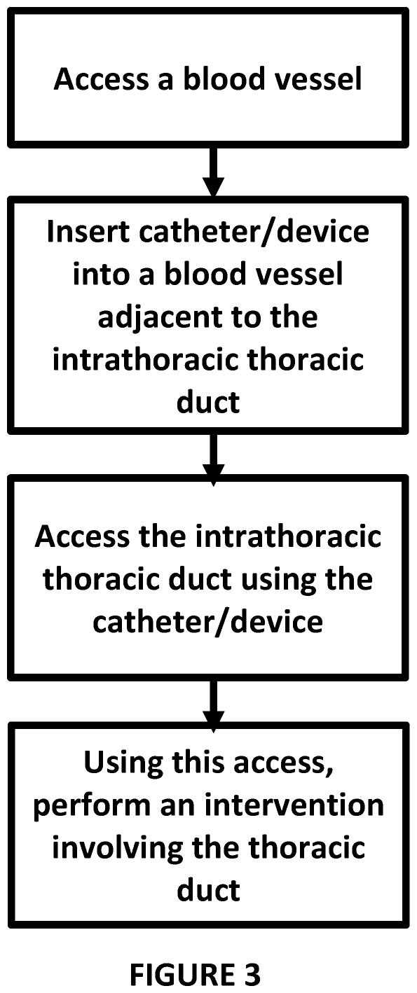

One implementation of intervening on the thoracic duct is shown in FIG. 3 and FIG. 6C. After accessing the thoracic duct, for example as illustrated in FIG. 2 and FIG. 6B, the access catheter device 610 can be removed leaving the guidewire 620 in place. A catheter 630 can be advanced over the guidewire 620, thus traveling from external to the patient, into the azygos vein 640, and then into the thoracic duct 650. This catheter can be used for withdrawal of lymphatic fluid/chyle and/or infusion of medications and/or devices such as embolization materials. In an additional implementation, the catheter 630 can comprise a stent catheter to place a stent or other device intended to connect the thoracic duct to the azygos vein, which can shunt at least a portion of lymph from the thoracic duct 650 to the azygos vein 640. In an additional implementation, the catheter 630 can be a shunt creation catheter to create a shunt between the thoracic duct 650 and the azygos vein 640 using energy such as thermal energy.

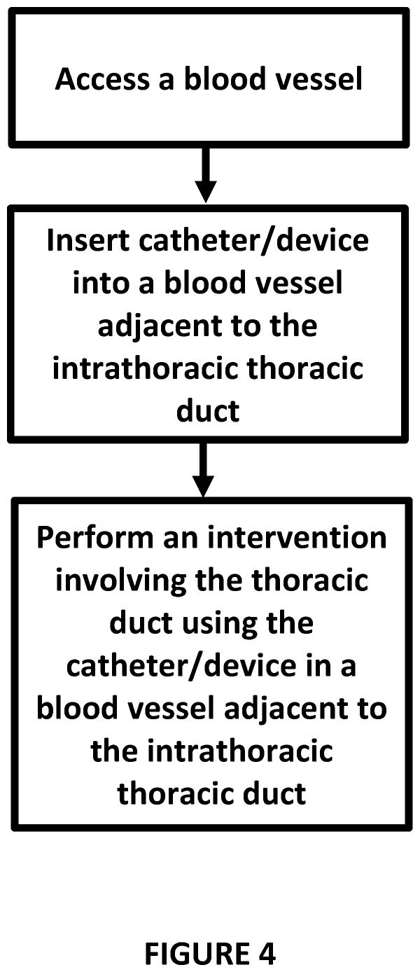

An additional implementation of intervening on the thoracic duct is illustrated in FIG. 4. After accessing a blood vessel, for example though not limited to limited to percutaneously into the right internal jugular vein, a catheter/device is advanced into a vein adjacent to the thoracic duct. For example, a catheter/device can be advanced over a guidewire that was placed into the azygos vein adjacent to the thoracic duct. After catheter/device placement, intervention on the thoracic duct can be performed, for example as described previously without first placing a guidewire into the thoracic duct.

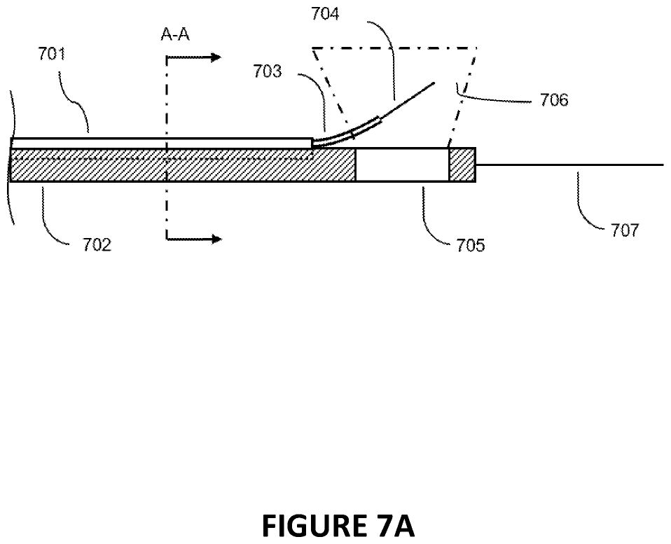

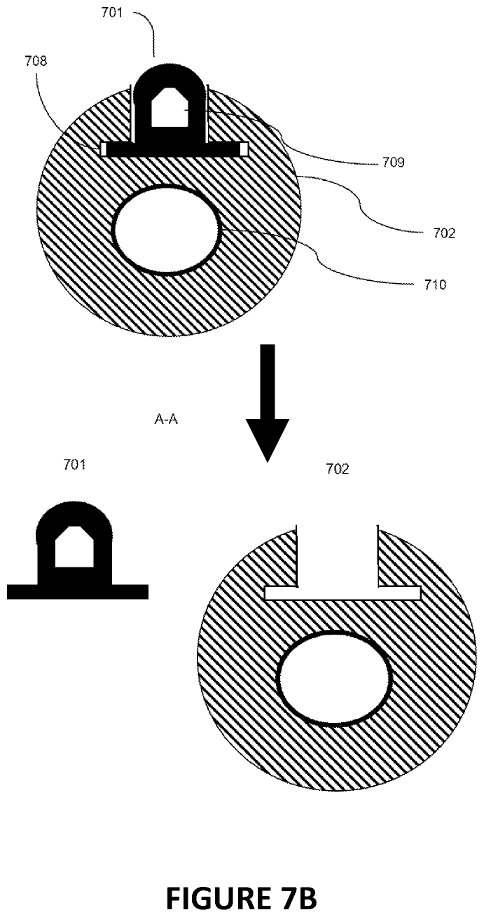

See for example implementations of the present invention shown in FIGS. 7A and 7B, 8A and 8B, and 9A and 9B. In certain implementations, the thoracic duct is accessed with a needle, wherein the needle guide and intravascular ultrasound (IVUS) or intracardiac echocardiography (ICE) catheter can be separated from each other without having to remove the IVUS or ICE catheter. This is useful because the IVUS or ICE catheter can further be used for visualization when a catheter is advanced over the guidewire that goes into the thoracic duct.

Use of novel needle guide device/imaging catheter that are separably joined:

1. Access a blood vessel
2. Insert imaging catheter and needle guide device into a blood vessel adjacent to the thoracic duct/cisterna chyli
3. Access the thoracic duct/cisterna chyli using the needle guide device while visualizing with the imaging catheter
4. Insert a guidewire through the needle guide device into the thoracic duct/cisterna chyli
5. Separate the needle guide device from the imaging catheter
6. Remove the needle guide device from the body while leaving thoracic duct guidewire and imaging catheter in place
7. Perform intervention (e.g., insert a catheter over the guidewire into the thoracic duct)

Variation:

1. Access a blood vessel
2. Insert imaging catheter and needle guide device into a blood vessel adjacent to the thoracic duct/cisterna chyli
3. Access the thoracic duct/cisterna chyli using the needle guide device while visualizing with the imaging catheter
4. Insert a guidewire through the needle guide device into the thoracic duct/cisterna chyli
5. Remove needle guide device from body while leaving guidewire and imaging catheter in place, which are no longer joined
6. Perform intervention (e.g., insert a catheter over the guidewire into the thoracic duct)

One embodiment is shown in FIGS. 7A and 7B. Needle guide device 701 is slidably disposed with imaging catheter 702 through a formed slot that accommodates the shape of 701 and allows 701 and 702 to track through the vasculature as a single unit without significant axial separation. The shape of the slot 708 and the cross-sectional shape of 701 can be such that they limit rotation as to keep the imaging field 706, curved needle 703, and guidewire through curved needle 704 in a similar plane for improved visualization of the needle, guidewire, and thoracic duct. A cross section of the system indicated by A-A demonstrates the interface between 701 and 702 with slot 708. The slot 708 can accommodate the entire length of needle guide device 701 or a portion of it, such as just the distal end of the needle guide device. Needle guide device 701 allows the passage of the curved needle 703 therethrough. Guidewire 704 can fit through curved needle 703. Once the thoracic duct is accessed, the needle guide device 701 and/or curved needle 703 can be longitudinally withdrawn from imaging device 702 such that the guidewire 704 is no longer axially connected to imaging device 702. This allows for further interventions to be performed while maintaining imaging catheter 702 in place.

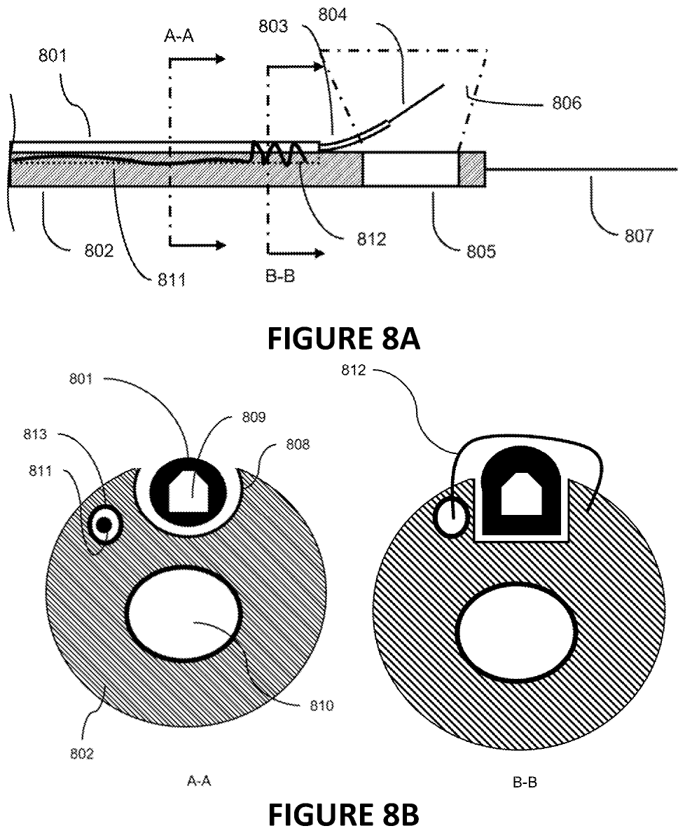

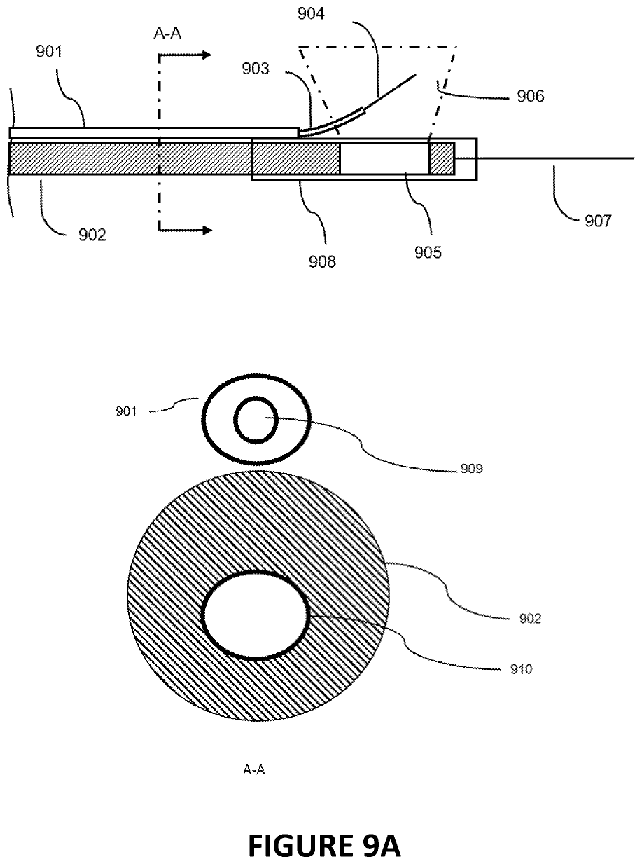

Another embodiment shown in FIGS. 8A and 8B where needle guide device 801 and imaging catheter 802 are held together at least in part by a removable fiber 811 running through imaging catheter 802 and wrapped or tied as 812 around the distal end of needle guide device 801. Fiber 811 runs alongside or through imaging catheter 802 such as in a hole 813, exiting catheter 802 and wrapping around needle guide device 801 as fiber 812 at least once to prevent the needle guide device 801 from separating from imaging catheter 802 prematurely. The distal end of needle guide device 801 can key into 802 using geometric shapes such that imaging field 806 and curved needle 803 are in same plane as shown in cross section B-B (FIG. 8B). Once the thoracic duct is accessed the fiber 811 can be pulled proximally, unwrapping or untying from around needle guide device 802, to release the needle guide device 801 from imaging catheter 802.
801: needle guide device
802: imaging catheter
803: curved needle to access thoracic duct
804: guidewire through curved needle
805: imaging catheter imaging array
806: imaging field
807: access guidewire
808: slot in imaging catheter for needle guide device
809: hole through needle guide device for curved needle
810: hole through imaging device for access guidewire
811: fiber running through 802
812: fiber wrapping around 801
813: longitudinal hole through 802 for fiber 811

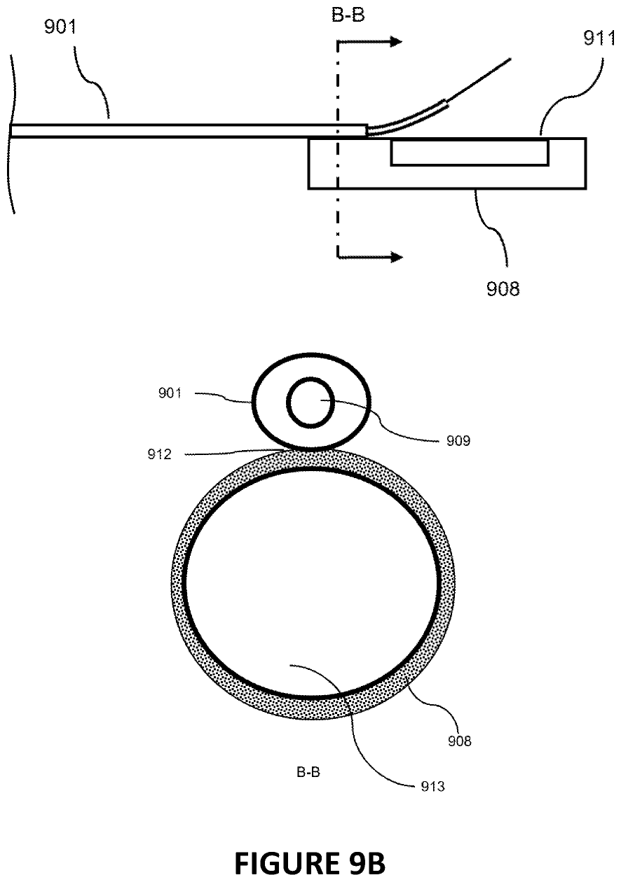

Another embodiment shown in FIGS. 9A and 9B. 901 needle guide device includes a distal portion 908 that fits over an imaging catheter 902 such that 901 can be sold as a kit that can be inserted/connected to existing 902. 908 can key into 902 using geometric shapes such that imaging field 906 and curved needle 903 are in same plane. Needle guide device 901 is connected to distal portion 908 at 912. A cross section of the system at plane A-A is shown in FIG. 9A and a cross section of the distal portion of needle guide device 901 B-B is shown in FIG. 9B.

Certain implementations are provided in FIG. 7A, FIG. 7B, FIG. 8A, FIG. 8B, FIG. 9A, and FIG. 9B, and described herein, and reference can be made to the following reference numerals:

FIGS. 7A and 7B
701: needle guide device
702: imaging catheter
703: curved needle to access thoracic duct
704: guidewire through curved needle
705: imaging catheter imaging array
706: imaging field
707: access guidewire
708: slot in imaging catheter for needle guide device
709: hole through needle guide device for curved needle
710: hole through imaging device for access guidewire

701 & 703 can be removed/withdrawn through 708 after accessing the thoracic duct, leaving the 704 guidewire in the thoracic duct and freed from imaging catheter 702.

[RESERVED]

FIGS. 8A and 8B:
801: needle guide device
802: imaging catheter
803: curved needle to access thoracic duct
804: guidewire through curved needle
805: imaging catheter imaging array
806: imaging field
807: access guidewire
808: slot in imaging catheter for needle guide device
809: hole through needle guide device for curved needle
810: hole through imaging device for access guidewire
811: fiber running through 802
812: fiber wrapping around 801
813: longitudinal hole through 802 for fiber 811

Once guidewire 804 is in thoracic duct, pull on mechanism attached to fiber 811 to unwrap/unravel 812 from around 801, thereby releasing 801 from 802

[RESERVED]

FIGS. 9A and 9B:
901: needle guide device—full length
902: imaging catheter
903: curved needle to access thoracic duct
904: guidewire through curved needle
905: imaging catheter imaging array
906: imaging field
907: access guidewire
908: distal portion of 901 needle guide device
909: hole through needle guide device for curved needle
910: hole through imaging device for access guidewire
911: slot in 908 to allow for imaging device field of view
912: junction between 901 and 908
913: opening for imaging device 902 to fit into

901 needle guide device includes a distal portion 908 that fits over an imaging catheter such that 901 can be sold as a kit that can be inserted/connected to existing 902. 908 can key into 902 using geometric shapes such that imaging field 906 and curved needle 903 are in same plane

[RESERVED]

As is evident from the foregoing description, and the Examples set forth above and below in detail, certain aspects of the present technology as disclosed are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the scope of the present technology as disclosed and claimed.

Other aspects, objects and advantages of the present technology as disclosed can be obtained from a study of the drawings, the disclosure and the appended claims.

[RESERVED]

[RESERVED]

### EXAMPLES

### Example 1

Implementations:

1. A method, comprising:

Accessing a blood vessel;

Advancing an intravascular imaging device through a vasculature into a vein;

Imaging a thoracic duct and/or a cisterna chyli using the intravascular imaging device.

2. The method of implementation 1, wherein the vein comprises an azygos vein, a hemiazygos vein, a tributary of an azygos vein, or a tributary of a hemiazygos vein.
3. The method of implementation 1, wherein the blood vessel comprises an internal jugular vein.
4. The method of implementation 1, wherein the imaging device comprises an intravascular ultrasound device.
5. The method of implementation 1, wherein the imaging device comprises an optical coherence tomography device.
6. A method, comprising:

Accessing a blood vessel;

Advancing a medical device through the vasculature into a vein;

Accessing a thoracic duct and/or a cisterna chyli from the vein using the medical device.

7. The method of implementation 6, wherein the vein comprises an azygos vein, a hemiazygos vein, a tributary of an azygos vein, or a tributary of a hemiazygos vein.
8. The method of implementation 6, wherein the blood vessel comprises an internal jugular vein.
9. The method of implementation 6, wherein the medical device comprises an imaging device.
10. The method of implementation 9, wherein the imaging device comprises an integrated intravascular ultrasound device.
11. The method of implementation 9, wherein the medical device further comprises an access device.
12. The method of implementation 11, wherein the access device comprises a needle.
13. The method of implementation 6, wherein the medical device comprises an access device.
14. The method of implementation 13, wherein the access device comprises a needle.
15. The method of implementation 6, wherein the medical device comprises at least one imaging device and at least two access devices.
16. The method of implementation 6, wherein the medical device comprises at least two imaging devices, and at least one access device.
17. The method of implementation 6, wherein the medical device comprises at least two imaging devices and at least two access devices.
18. The method of implementation 6, further comprising advancing a guidewire into the thoracic duct and/or cisterna chyli.
19. The method of implementation 14, wherein the needle becomes a guidewire after introduction into the thoracic duct and/or cisterna chyli.
20. The method of implementation 18, further comprising advancing a catheter over the guidewire into the thoracic duct and/or cisterna chyli.
21. The method of implementation 19, further comprising advancing a catheter over the guidewire into the thoracic duct and/or cisterna chyli.
22. The method of implementation 20, further comprising withdrawing fluid and/or cells via the catheter.
23. The method of implementation 21, further comprising withdrawing fluid and/or cells via the catheter.
24. The method of implementation 20, further comprising instilling fluid, medicine, and/or cells into the thoracic duct and/or cisterna chyli.
25. The method of implementation 1, wherein the vein is an azygos vein, a hemiazygos vein, a tributary of an azygos vein, or a tributary of a hemiazygos vein.
26A. A method of diagnosing a thoracic duct and/or thoracic duct pathology comprising:

inserting an intravascular imaging device into a blood vessel adjacent to the thoracic duct,

imaging the intrathoracic thoracic duct with the intravascular imaging device

26B. A method of treatment comprising

inserting an intravascular imaging device into a blood vessel adjacent to the thoracic duct,

imaging the intrathoracic thoracic duct with the intravascular imaging device
27. A method of accessing the thoracic duct comprising:

inserting a catheter/device into a blood vessel adjacent to the thoracic duct,

accessing the intrathoracic thoracic duct using this catheter/device.

28. A method of performing a medical intervention comprising:

inserting a catheter/device into a blood vessel adjacent to a thoracic duct,

accessing the intrathoracic thoracic duct using the catheter/device,

performing an intervention involving the thoracic duct using this intrathoracic thoracic duct access,

29. A method of performing a medical intervention comprising:

inserting a catheter/device into a blood vessel adjacent to a thoracic duct,

performing an intervention involving the thoracic duct using the catheter/device in the blood vessel adjacent to the intrathoracic thoracic duct.

31. A method, comprising:

Accessing a blood vessel;

Advancing an intravascular imaging device through the blood vessel into a vein, wherein the vein is an azygos vein, a hemiazygos vein, a tributary of an azygos vein, or a tributary of a hemiazygos vein;

Imaging a thoracic duct and/or a cisterna chyli using the intravascular imaging device.

32. The method of implementation 31, wherein the blood vessel is an internal jugular vein.
33. The method of implementation 31, wherein the imaging device comprises an intravascular ultrasound device.
34. The method of implementation 31, wherein the imaging device comprises an optical coherence tomography device.
35. A method, comprising:

Accessing a blood vessel;

Advancing a medical device through the blood vessel into a vein, wherein the vein is an azygos vein, a hemiazygos vein, a tributary of an azygos vein, or a tributary of a hemiazygos vein;

Accessing a thoracic duct and/or a cisterna chyli from the vein using the medical device.

36. The method of implementation 35, wherein the blood vessel is an internal jugular vein.
37. The method of implementation 35, wherein the medical device comprises an imaging device.
38. The method of implementation 37, wherein the imaging device comprises an integrated intravascular ultrasound device.
39. The method of implementation 37, wherein the medical device further comprises an access device.
40. The method of implementation 39, wherein the access device comprises a needle.
41. The method of implementation 35, wherein the medical device comprises an access device.
42. The method of implementation 41, wherein the access device comprises a needle.
43. The method of implementation 35, wherein the medical device comprises at least one imaging device and at least two access devices.
44. The method of implementation 35, wherein the medical device comprises at least two imaging devices, and at least one access device.
45. The method of implementation 35, wherein the medical device comprises at least two imaging devices and at least two access devices.
46. The method of implementation 35, further comprising advancing a guidewire into the thoracic duct and/or cistema chyli.
47. The method of implementation 42, wherein the needle becomes a guidewire after introduction into the thoracic duct and/or cisterna chyli.
48. The method of implementation 46, further comprising advancing a catheter over the guidewire into the thoracic duct and/or cistema chyli.
49. The method of implementation 47, further comprising advancing a catheter over the guidewire into the thoracic duct and/or cisterna chyli.
50. The method of implementation 48, further comprising withdrawing fluid and/or cells via the catheter.
51. The method of implementation 49, further comprising withdrawing fluid and/or cells via the catheter.
52. The method of implementation 48, further comprising instilling fluid, medicine, and/or cells into the thoracic duct and/or cistema chyli.
53. A kit comprising instructions according to any of Implementations 31-52:
54. Instructions for a procedure comprising steps according to any of Implementations 31-52
55. A Label of instructions for a procedure comprising steps according to any of Implementations 31-52
56. A device for accessing a thoracic duct in a patient, comprising:

a needle guide device, comprising a curved needle to access the thoracic duct and a guidewire through the curved needle;

an imaging catheter, comprising an access guidewire, an imaging array, and a slot for receiving the needle guide device;

57. The device of implementation 56, wherein the imaging catheter is a removable imaging catheter and wherein the needle guide device is separable from the imaging catheter.
57. A method for accessing a thoracic duct in a patient in need thereof, comprising:

Accessing a blood vessel;

Advancing a medical device through the blood vessel into a vein, wherein the vein is an azygos vein, a hemiazygos vein, a tributary of an azygos vein, or a tributary of a hemiazygos vein;

Accessing a thoracic duct and/or a cisterna chyli from the vein using the medical device, wherein the medical device comprises:
a needle guide device, comprising a curved needle to access the thoracic duct and a guidewire through the curved needle, and
an imaging catheter, comprising an access guidewire, an imaging array, and a slot for receiving the needle guide device;
   58. The method of implementation 57, wherein the imaging catheter is a removable imaging catheter and wherein the needle guide device is separable from the imaging catheter.

[RESERVED]

### Example 2

Certain useful implementations of the invention utilizing the azygos vein, hemiazygos vein, tributary of the azygos vein, or tributary of the hemiazygos vein to visualize/access/treat the thoracic duct, including devices, systems, and methods:

Diagnostic imaging of the thoracic duct
With intravascular ultrasound
With optical coherence tomography

Injecting contrast (including but not limited to iodinated contrast, gadolinium) into the thoracic duct for diagnostic imaging

Removing lymphatic fluid
As a method of treatment for heart failure
As a method for immunosuppression
As a method of treatment for edema
As a method of treatment for ascites
As a method of treatment for chyle/lymph leak
As a method of treatment for sepsis
As a method of treatment for toxic ingestion
As a method of treatment for lymphatic metastatic disease
As a method of diagnosing metastatic disease
As a method of diagnosing disorders involving abnormalities in the lympathic fluid/chyle

Instilling fluid, devices, and/or materials into the thoracic duct
Medications: antibiotics, antifungals, antivirals, immunosuppressants, anti-inflammatories, chemotherapy, pain control medications, etc.
Treated lymphatic fluid
   Lymphatic fluid/chyle after removing cellular contents
   Lymphatic fluid/chyle after removing inflammatory proteins such as cytokines
Cells
   Immune cells
   Stem cells
Lymphatic fluid from same individual or separate individual
Glue (such as n-butyl cyanoacrylate), plugs, coils, hydrogel, sclerosant, foam, Onyx or other embolization material/sclerosant
Stents
Stent grafts
As a method of treatment for:
   Chyle leak
   Lymphatic leak
   Protein losing enteropathy
   Plastic bronchitis
   Thoracic duct stenosis
   Edema
   Heart failure
   Ascites

Create a shunt between thoracic duct and venous system
With a mechanical shunt device (such as a stent or flared disc with patent center) between the thoracic duct and vein
Via thermal means create a connection between thoracic duct and vein
Via mechanical means (such as stitching) create a connection between thoracic duct and vein
As a method of treatment for:
   Ascites
   Heart failure
   Thoracic duct stenosis/obstruction
   Chyle leak
   Lymphatic leak

Ablate the thoracic duct
RF ablation
Cryoablation
Microwave ablation

[RESERVED]

[RESERVED]

What is claimed is:

1. A method, comprising:
Accessing a blood vessel;
Advancing an intravascular imaging device through the blood vessel into a vein, wherein the vein is an azygos vein, a hemiazygos vein, a tributary of an azygos vein, or a tributary of a hemiazygos vein;
Imaging a thoracic duct and/or a cisterna chyli using the intravascular imaging device.

2. The method of claim 1, wherein the blood vessel is an internal jugular vein.

3. The method of claim 1, wherein the imaging device comprises an intravascular ultrasound device.

4. A method, comprising:
Accessing a blood vessel;
Advancing a medical device through the blood vessel into a vein, wherein the vein is an azygos vein, a hemiazygos vein, a tributary of an azygos vein, or a tributary of a hemiazygos vein;
Accessing a thoracic duct and/or a cisterna chyli from the vein using the medical device.

5. The method of claim 4, wherein the blood vessel is an internal jugular vein.

6. The method of claim 4, wherein the medical device comprises an imaging device.

7. The method of claim 6, wherein the imaging device comprises an integrated intravascular ultrasound device.

8. The method of claim 6, wherein the medical device further comprises an access device.

9. The method of claim 8, wherein the access device comprises a needle.

10. The method of claim 4, wherein the medical device comprises an access device.

11. The method of claim 10, wherein the access device comprises a needle.

12. The method of claim 4, further comprising advancing a guidewire into the thoracic duct and/or cisterna chyli.

13. The method of claim 11, wherein the needle further comprises a guidewire after introduction into the thoracic duct and/or cistema chyli.

14. The method of claim 12, further comprising advancing a catheter over the guidewire into the thoracic duct and/or cistema chyli.

15. The method of claim 13, further comprising advancing a catheter over the guidewire into the thoracic duct and/or cistema chyli.

16. The method of claim 14, further comprising withdrawing fluid and/or cells via the catheter.

17. The method of claim 15, further comprising withdrawing fluid and/or cells via the catheter.

18. The method of claim 14, further comprising instilling fluid, medicine, and/or cells into the thoracic duct and/or cistema chyli.

19. A device configured for accessing a thoracic duct, comprising
an elongated body comprising a proximal end portion functionally connected to a distal end portion;
wherein the distal end portion is configured to access a blood vessel and traverse an intravascular space of the blood vessel;
wherein the distal end portion is further configured to access a vein, wherein the vein is an azygos vein, a hemiazygos vein, a tributary of an azygos vein, or a tributary of a hemiazygos vein;
wherein the distal end portion is further configured to traverse the vein and pass through a wall of the vein, traverse a potential space between the vein and into a thoracic duct and/or a cisterna chyli.

20. The device of claim 19, wherein said device comprises a separably connected imaging catheter and needle guide device.

21. The device of claim 20, wherein
the needle guide device comprises a curved needle to access the thoracic duct and/or a cisterna chyli, and a guidewire through the curved needle;
the imaging catheter comprises an access guidewire, an imaging array, and a slot for receiving the needle guide device.

22. The device of claim 20, wherein the proximal end portion is configured to receive fluid and/or cells via the distal end portion.

23. The device of claim 20, wherein the distal end portion is configured to instill fluid, medicine, and/or cells into the thoracic duct and/or cisterna chyli.
